SEQUENCE LISTING (10 sequences)

SEQ ID NO: 1 - protein, 11 aa, Human immunodeficiency virus
YGRKKRRQRRR

SEQ ID NO: 2 - DNA, 827 bp, Homo sapiens
  CDS (120)..(632)
gcagtggaggcggcccaggcccgccttccgcagggtgtcgccgctgtgccgctagcggtgccccgcctgctgcggtggcaccagccaggaggcggagtggaagtggccgtggggcgggtatgggactagctggcgtgtgcgccctgagacgctcagcgggctatatactcgtcggtggggccggcggtcagtctgcggcagcggcagcaagacggtgcagtgaaggagagtgggcgtctggcggggtccgcagtttcagcagagccgctgcagccatggccccaatcaaggtgggagatgccatcccagcagtggaggtgtttgaaggggagccagggaacaaggtgaacctggcagagctgttcaagggcaagaagggtgtgctgtttggagttcctggggccttcacccctggatgttccaaggttcggctcctggctgatcccactggggcctttgggaaggagacagacttattactagatgattcgctggtgtccatctttgggaatcgacgtctcaagaggttctccatggtggtacaggatggcatagtgaaggccctgaatgtggaaccagatggcacaggcctcacctgcagcctggcacccaatatcatctcacagctctgaggccctgggccagattacttcctccacccctccctatctcacctgcccagccctgtgctggggccctgcaattggaatgttggccagatttctgcaataaacacttgtggtttgcggccaaaaaaaaaaaaaaaaaaaaaaaaaaaaaaaaaaaaaaaaaaaaaaaaaaaaaaaaaaaaaaaaaaaaaaaaaaaa

SEQ ID NO: 3 - protein, 170 aa, Homo sapiens
MGLAGVCALRRSAGYILVGGAGGQSAAAAARRCSEGEWASGGVRSFSRAAAAMAPIKVGDAIPAVEVFEGEPGNKVNLAELFKGKKGVLFGVPGAFTPGCSKVRLLADPTGAFGKETDLLLDDSLVSIFGNRRLKRFSMVVQDGIVKALNVEPDGTGLTCSLAPNIISQL

SEQ ID NO: 4 - DNA, 1715 bp, Homo sapiens
  CDS (52)..(726)
gaaccaaccggttgcttgctgtcccagcggcgccccctcatcaccgtcgccatgcccggaggtctgcttctcggggacgtggctcccaactttgaggccaataccaccgtcggccgcatccgtttccacgactttctgggagactcatggggcattctcttctcccaccctcgggactttaccccagtgtgcaccacagagcttggcagagctgcaaagctggcaccagaatttgccaagaggaatgttaagttgattgccctttcaatagacagtgttgaggaccatcttgcctggagcaaggatatcaatgcttacaattgtgaagagcccacagaaaagttaccttttcccatcatcgatgataggaatcgggagcttgccatcctgttgggcatgctggatccagcagagaaggatgaaaagggcatgcctgtgacagctcgtgtggtgtttgtttttggtcctgataagaagctgaagctgtctatcctctacccagctaccactggcaggaactttgatgagattctcagggtagtcatctctctccagctgacagcagaaaaaagggttgccaccccagttgattggaaggatggggatagtgtgatggtccttccaaccatccctgaagaagaagccaaaaaacttttcccgaaaggagtcttcaccaaagagctcccatctggcaagaaatacctccgctacacaccccagccttaagtctcttggagaagctggtgctgtgagccagaggatgtcagctgccaattgtgttttcctgcagcaattccataaacacatcctggtgtcatcacagccaaggtttttaggttgctataccaatggcttattaaatgaaaatggcactaaaagtttcttgagattctttatactctctgccttcagcaatcaattccattcatacatcagcactctgctggttctgtttgaaatatgttctgtatttaaaactcaaatcttgttggatctctgcagggcttgtgaccaatgaagtcatatttgttgatggttgacaaagcttgcttcactccatcagagaatgactatcaatttttttttaactgtcctatcacgtcctctcctgtcacccattttgaagagtggcagaacttgaagttcaacttcctctgtaaatatccaagtataaagcccaggaacttctagaataacccagatgcgctttaattttttttaatatgttttgatcacagaacttctagaataacccagatgctctttcatattcttttaatacatcttgatcacagctgggggaaaaaaagctttttaattctataccttcctagtagataagtgaagagcagggaaagagacctttaaatattttgctataaaaaaatttgtgataagtttctatcaaaatggggagattgcagaaaaggcttcccttggctcccaaggaggtgtagcaggtgtgagcaatattagtgccatgtgcctttcacacagggtttgcatttatcagtctgttttccgatgatgtgtacatgaaagagtacaccatgtgaagagaagagagaatgattgaaaatgttttagtatagaactcttcttgcagtgggttgctattttctagattttactttttagggaacaaaataaaatcctttgttaaaactgggaaaaaaaaaaaaaaaaaaaaaaaaaaaaaaaaaaaaaaaaaaaaa

SEQ ID NO: 5 - protein, 224 aa, Homo sapiens
MPGGLLLGDVAPNFEANTTVGRIRFHDFLGDSWGILFSHPRDFTPVCTTELGRAAKLAPEFAKRNVKLIALSIDSVEDHLAWSKDINAYNCEEPTEKLPFPIIDDRNRELAILLGMLDPAEKDEKGMPVTARVVFVFGPDKKLKLSILYPATTGRNFDEILRVVISLQLTAEKRVATPVDWKDGDSVMVLPTIPEEEAKKLFPKGVFTKELPSGKKYLRYTPQP

SEQ ID NO: 6 - DNA, 33 bp, Human immunodeficiency virus
tacggccgcaagaaacgccgccagcgccgccgc

SEQ ID NO: 7 - DNA, 25 bp, Artificial Sequence
  sense primer
gtcgccatggccggaggtctgcttc

SEQ ID NO: 8 - DNA, 26 bp, Artificial Sequence
  antisense primer
aattggcagctgacatcctctggctc

SEQ ID NO: 9 - DNA, 30 bp, Artificial Sequence
  sense primer
gctggtaccatggccccaatcaaggtggga

SEQ ID NO: 10 - DNA, 29 bp, Artificial Sequence
  antisense primer
tagaattcagagctgtgagatgatattgg

(19) United States
(12) Patent Application Publication
(10) Pub. No.: US 2011/0229449 A1
(43) Pub. Date: Sep. 22, 2011

(54) PROPHYLAXIS AND TREATMENT OF MACULAR DEGENERATION AND RETINOPATHY USING A PRDX PROTEIN

(75) Inventors: Eri Kubo, Fukui (JP); Dhirendra Pratap Singh, Omaha, NE (US)

(73) Assignee: National University Corporation University of Fukui, Fukui-shi, Fukui (JP)

(21) Appl. No.: 13/121,911

(22) PCT Filed: Mar. 4, 2010
(86) PCT No.: PCT/JP2010/054072

§ 371 Date: Jun. 8, 2011

Related U.S. Application Data
(60) Provisional application No. 61/157,815, filed on Mar. 5, 2009.

(51) Int. Cl. A61K 38/44; C12N 9/08; C07H 21/00; C12N 15/63; A61K 31/7088; A61P 27/02

(52) U.S. Cl. 424/94.4; 435/192; 536/23.2; 435/320.1; 514/44 R

(57) ABSTRACT

The present invention provides an agent for the prophylaxis or treatment of retinopathy or AMD containing PRDX 5 or PRDX 6, as well as an agent for the prophylaxis or treatment of retinopathy or AMD containing a polynucleotide encoding PRDX 5 or PRDX 6. The present invention also provides a method for the prophylaxis or treatment of retinopathy or AMD in a patient suffering from the disease, which includes administering to the patient an effective amount of PRDX 5 or PRDX 6, or administering to the patient an effective amount of a polynucleotide encoding PRDX 5 or PRDX 6.

### TECHNICAL FIELD

The present invention relates to an agent for the prophylaxis or treatment of retinopathy or age-related macular degeneration (hereinafter also referred to as AMD or macular degeneration), and a method for the prophylaxis or treatment of retinopathy or AMD.

### BACKGROUND ART

Hyperglycemia-induced oxidative stress is known to play a critical role in the development and progression of diabetic retinopathy (Baynes, 1991, Diabetes. 40, 405-412; Kowluru et al., 1998, Diabetes. 47, 464-469; Kowluru et al., 2001, Diabetes. 50, 1938-1942). Studies have shown that as retinal pericytes undergo oxidative stress, several molecular and biochemical changes under high-glucose conditions eventually cause apoptosis and accelerated cell death (Amano et al., 2005, Microvasc Res. 69, 45-55; Kowluru, 2003, Diabetes. 52, 818-823; Lorenzi and Gerhardinger, 2001, Diabetologia. 44, 791-804). Clinical and experimental data demonstrate that the generation of reactive oxygen species (ROS) is increased in both types of diabetes (Lorenzi and Gerhardinger, 2001, supra; Rosen et al., 2001, Diabetes Metab Res Rev. 17, 189-212; Setter et al., 2003, Ann Pharmacother. 37, 1858-1866). ROS production mediated by hyperglycemia is thought to be generated via a number of mechanisms including auto-oxidation of glucose, non-enzymatic glycation of proteins, glucose-induced activation of protein kinase C, increased polyol pathway activity, and the impaired anti-oxidant enzymes and alterations in mitochondria (Chung et al., 2003, J Am Soc Nephrol. 14, S233-236; Inoguchi et al., 2003, J Am Soc Nephrol. 14, S227-232; Kanwar et al., 2007, Invest Ophthalmol Vis Sci. 48, 3805-3811; Nishikawa et al., 2000, Nature. 404, 787-790; Sakurai and Tsuchiya, 1988, FEBS Lett. 236, 406-410).

Peroxiredoxins (PRDXs), a new family of antioxidants, function in connect to detoxify reactive oxygen species (ROS) and thus provide cytoprotection from internal/external environmental stress (Peshenko et al., 2001, J Ocul Pharmacol Ther. 17, 93-99; Wood et al., 2003b, Trends Biochem Sci. 28, 32-40). The mammalian PRDX family is composed of six members (PRDXs 1-6) (Fatma et al., 2001, J Biol Chem. 276, 48899-48907; Lyu et al., 1999, Mamm Genome. 10, 1017-1019; Wood et al., 2003a, Science. 300, 650-653; Wood et al., 2003b, supra). All PRDXs have two catalytically active cysteines, except PRDX 6, a cytosolic antioxidant protein, which contains only one (Fatma et al., 2001, supra; Lyu et al., 1999, supra; Wood et al., 2003a, supra; Wood et al., 2003b, supra). PRDX 5 is a novel and unusual peroxiredoxin with mitochondrial and peroxisomal targeting signals (Verdoucq et al., 1999, J Biol Chem. 274, 19714-19722; Zhou et al., 2000, Biochem Biophys Res Commun. 268, 921-927) and characterized as a thioredoxin peroxidase. PRDX 6 has been documented to inhibit peroxynitrite (Peshenko and Shichi, 2001, Free Radic Biol Med. 31, 292-303; Peshenko et al., 2001, supra) and phospholipid hydroperoxide reductase activities (Chen et al., 2000, J Biol Chem. 275, 28421-28427; Manevich et al., 2002, Proc Natl Acad Sci USA. 99, 11599-11604).

High glucose levels attenuate the function of pericytes through the generation of free radicals, suggesting a possible pathophysiological linkage to diabetic retinopathy with the oxidant-antioxidant balance. Our previous study revealed decreased expression of PRDX 6 mRNA and protein in rat lenses with diabetes or galactose-induced cataracts (Kubo et al., 2005, Diabetologia. 48, 790-798.; Kubo et al., 2004, Biochem Biophys Res Commun. 314, 1050-1056). Hyperglycemia causes a decrease of this protein in lens epithelial cells (LECs) and the cells subsequently undergo apoptosis (Kubo et al., 2005, Diabetologia. 48, 790-798; Kubo et al., 2004, supra). The present inventors hypothesized that PRDXs may be able to remove H₂O₂ or capture ROS in pericytes under hyperglycemic conditions thereby protecting the cells from hyperglycemia-induced pericyte loss.

Additionally, several studies support the hypothesis that oxidative damage to the retinal pigment epithelial cells (RPE) may play a role in some of the key features of age-related macular degeneration (AMD) (Voloboueva et al., 2007, FASEB J. 21(14), 4077-4086; Justilien et al., 2007; Invest Ophthalmol Vis Sci. 48(10), 4407-4420). However, the effectiveness of PRDX 5 or PRDX 6 in the prophylaxis or treatment of retinopathy or macular degeneration had not been clarified.

### SUMMARY OF THE INVENTION

It is therefore an object of the present invention to provide a novel agent which is useful for the prophylaxis or treatment of retinopathy or AMD, and a method for the prophylaxis or treatment of retinopathy or AMD.

Advances in gene/protein delivery, and identification of several protein transduction domains (PTDs) has made possible delivery of proteins to cells or organs (Frankel and Pabo, 1988, Cell. 55, 1189-1193; Green and Loewenstein, 1988, Cell. 55, 1179-1188). HIV-Trans-Activating Transduction (TAT) domain has 11 amino acids (aa; YGRKKRRQRRR (SEQ ID NO:1)) and has 100% potential for intracellular delivery of proteins across the plasma membrane and the blood brain barrier (Becker-Hapak et al., 2001, Methods. 24, 247-256; Kubo et al., 2008, Am J Physiol Cell. 294, C842-C855; Mann and Frankel, 1991, Embo J. 10, 1733-1739; Nagahara et al., 1998, Nat Med. 4, 1449-1452; Rusnati et al., 1997, J Biol Chem. 272, 11313-11320). The present inventors previously demonstrated that this recombinant PRDX 6 protein linked to TAT is internalized into LECs and is biologically active (Kubo et al., 2008, supra). Taking advantage of the ability of TAT domain to reach into cells, in the present invention, recombinant TAT-linked PRDX 5 and 6 proteins were used to assess those abilities in protecting pericytes against hyper-glucose-induced oxidative stress.

To achieve the above-mentioned object, the present inventors have tried to examine the effects of adding PRDXs 5 and 6 on high-glucose-induced cell death and oxidative stress using pig pericytes. The present inventors' aim was to provide new information on the antioxidant defenses against pericyte loss in diabetic retinopathy and efficacy of PRDX 5 and 6 proteins in diabetic pericyte loss. The present inventors also aimed to provide new information on the antioxidant defenses and efficacy of PRDX 5 and 6 proteins in retinal pigment epithelial cells (RPE) relating to AMD.

The present inventors have conducted further investigations based on these findings, and completed the present invention.

Accordingly, the present invention provides:

[1] An agent for the prophylaxis or treatment of retinopathy or macular degeneration, comprising a PRDX family protein;

[2] The agent of above [1], wherein the retinopathy is diabetic retinopathy;

[3] The agent of above [1] or [2], wherein the PRDX family protein is PRDX 5 or PRDX 6;

[4] The agent of any one of above [1] to [3], wherein the PRDX family protein is fused with a protein transduction domain (PTD);

[5] A method for preventing or treating retinopathy or macular degeneration, comprising administrating an effective, amount of a PRDX family protein to a subject in need thereof;

[6] The method of above [5], wherein the retinopathy is diabetic retinopathy;

[7] The method of above [5] or [6], wherein the PRDX family protein is PRDX 5 or PRDX 6;

[8] The method of any one of above [5] to [7], wherein the PRDX family protein is fused with a PTD;

[9] Use of a PRDX family protein for the manufacture of an agent for the prophylaxis or treatment of retinopathy or macular degeneration;

[10] An agent for the prophylaxis or treatment of retinopathy or macular degeneration, comprising a polynucleotide encoding a PRDX family protein;

[11] The agent of above [10], wherein the retinopathy is diabetic retinopathy;

[12] The agent of above [10] or [11], wherein the PRDX family protein is PRDX 5 or PRDX 6;

[13] The agent of any one of above [10] to [12], wherein the polynucleotide encodes a fusion protein of the PRDX family protein and PTD;

[14] The agent of any one of above [10] to [13], wherein the polynucleotide is incorporated into an expression vector;

[15] A method for preventing or treating retinopathy or macular degeneration, comprising administrating an effective amount of a polynucleotide encoding a PRDX family protein to a subject in need thereof;

[16] The method of above [15], wherein the retinopathy is diabetic retinopathy;

[17] The method of above [15] or [16], wherein the PRDX family protein is PRDX 5 or PRDX 6;

[18] The method of any one of above [15] to [17], wherein the polynucleotide encodes a fusion protein of the PRDX family protein and PTD;

[19] The method of any one of above [15] to [18], wherein the polynucleotide is incorporated into an expression vector;

[20] Use of a polynucleotide encoding a PRDX family protein for the manufacture of an agent for the prophylaxis or treatment of retinopathy or macular degeneration.

The characteristics and the advantages of the present invention will be apparent from the detailed description of the invention below.

### BRIEF DESCRIPTION OF THE DRAWINGS

FIG. 1 shows the high expression level of PRDXs 5 and 6 in rat retinas.

(A) Female, 7-week-old, Sprague-Dawley (SD) albino rats were used. Total RNA from rat retina (n=6) was isolated and transcribed into cDNA. Quantitative real-time PCR was carried out using specific primers. mRNA expression of each PRDX was adjusted to the mRNA copies of glyceraldehyde-3-phosphate dehydrogenase (GAPDH). Results indicated that mRNA expression level of PRDXs 5 and 6 were significantly high in comparison to other PRDXs (*p<0.0001). (B) Protein from female, SD rat retinas (7-week-old, n=8) was extracted and protein blot was performed using anti-PRDX 1-6 Abs. Protein blot for all six PRDXs revealed greater abundance of PRDX 5 and 6 proteins in rat retina, and the expression levels of PRDX 5 protein were higher than that of the other PRDXs. Results are derived from 4 different isolates (2 rats were used for each isolate) (*p<0.0001). (C) Immunohistochemical localization of PRDXs 5 and 6 and α-SMA (α-smooth muscle actin) in rat retina is shown. Seven-week-old, rat eyes (n=4) were paraffin-embedded and sectioned after being fixed in 4% paraformaldehyde, and sections were immunostained using Abs specific to PRDXs 5 and 6 and αSMA. Green color of positive staining of PRDX 5 (a) and PRDX 6 (b) was observed. Red color of positive staining of αSMA was localized in vessels suggesting that pericytes were visualized using anti-αSMA Ab (c). Hoechst stained nuclei were observed in blue color in each panels.

FIG. 2 shows the effect of D-glucose on expressions of PRDX 5 and 6 mRNA and proteins in cultured pig pericytes.

Pig pericytes were cultured in a medium containing 5.5 mM (5 G) or 30 mM (30 G) levels of D-glucose for 6 and 10 days. Four independent samples (n=4) were used in each treatment group. (A) Total RNA from pig pericyte was isolated and transcribed into cDNA. Real-time PCR was carried out using specific primers. mRNA expression of each PRDX was adjusted to the mRNA copies of ribosomal RNA. (B) Protein was extracted from pig pericyte and used for the protein blot. Cells cultured in high-glucose (30 G) medium showed apparent diminution of PRDX 5 mRNA (A; *p<0.005) and protein (B; *p<0.05) on the 10th day, while no change was detected in the expression of β-actin level, suggesting high glucose specifically modified the expression of PRDX 5 (A and B). However, the expression of PRDX 6 mRNA did not change on the 6th and 10th day in high (30 G) glucose medium groups in comparison to normal (5 G) glucose medium groups. Results are derived from 4 different isolates.

FIG. 3 shows transduction of TAT-HA-PRDX 5 and 6 into the pig pericytes.

Cells (2×10⁵ cells) were cultured in 60 mm plates. The next day, 5 μg/ml recombinant TAT-HA-PRDX 5 or 6 protein was added to the culture medium, and transduction of TAT-HA-PRDX 5 or 6 was assessed after 24 and 48 hours. Cells were washed, protein was extracted, and protein blot was performed using anti-HisG antibody (Invitrogen). Results revealed the intracellular transduction of TAT-HA-PRDXs 5 and 6 (lane; TAT) whereas (HA)-PRDXs 5 and 6 with flag tag (HA) only could not internalize into cells (lane; NC). Three experiments were done for each assay using 4 different isolates.

FIG. 4 shows the effect of PRDX 5 treatment on cellular survival of pig pericytes.

Cells were cultured in a medium containing 5.5 mM (5 G), 30 mM (30 G) or 50 mM (50 G) D-glucose supplemented with 10 μg/ml TAT-HA-PRDX 5 or bovine serum albumin for 6 and 10 days in 96 well-plates. Six or 10 days later, cell viability was estimated using colorimetric MTS assay. Addition of PRDX 5 significantly protected the inhibition of cell growth in the high-glucose (30 G and 50 G) medium at 6 and 10 days after culture. In hyperosmolar condition (30M; 5.5 mM glucose+24.5 mM mannitol, 50M; 5.5 mM glucose+44.5 mM mannitol,), there was no significant changes of cell viability in comparison to normal (5 G) group. *p<0.003; ** p<0.01; ***p<0.001; ****p<0.0005. Results were derived from 4 different samples.

FIG. 5 shows the effect of PRDX 5 and 6 treatments against high-glucose-induced apoptosis in pig pericytes.

Cells were cultured in a medium containing 5.5 mM (5 G) 30 mM (30 G) or 50 mM (50 G) D-glucose supplemented with 10 μg/ml TAT-HA-PRDX 5 or -PRDX 6 or bovine serum albumin as control for 6 and 10 days. Six or 10 days later, apoptotic cell death was estimated using TUNEL assay. On the 6th day after high-glucose culture, percentages of TUNEL positive cells were significantly increased in high glucose (50 G) medium group. On the 10th day after high-glucose culture, percentages of TUNEL positive cells were significantly increased in high glucose (30 G and 50 G) medium group. Addition of PRDXs 5 and 6 significantly inhibited apoptotic cell death in high-glucose-exposed pericytes. *p<0.0004; **p<0.00001; ***p<0.0007. Results were derived from 4-different samples.

FIG. 6 shows the effect of PRDX 5 and 6 treatment against oxidative stress-induced DNA damage in high-glucose-exposed pig pericytes.

Cells were cultured in a medium containing 5.5 mM (5 G) or 30 mM (30 G) D-glucose supplemented with 10 μg/ml TAT-HA-PRDX 5 or bovine serum albumin for 10 days. Ten days later, oxidative stress-induced DNA damage was estimated using immunolocalization of 8-OHdG. On the 10th day after high-glucose culture, 8-OHdG positive-pericytes (arrows) were significantly increased in comparison to control (5 G). Addition of PRDXs 5 and 6 significantly inhibited oxidative stress-induced DNA damage in high-glucose-exposed pericytes. *p<0.00001. Results were derived from 4 different samples.

FIG. 7 shows the effect of PRDXs 5 and 6 on RPE cell survival under H₂O₂-induced oxidative stress. PRDXs 5 and 6 showed suppressive effects against cultured RPE cell death in 200 μM H₂O₂.

FIG. 8 shows the expression of PRDXs in human cultured RPE. The expression of PRDXs 5 and 6 were relatively high in RPE.

### DETAILED DESCRIPTION OF THE INVENTION

The present invention provides an agent for the prophylaxis or treatment of retinopathy or macular degeneration, comprising a PRDX family protein.

The PRDX family protein of the present invention is a peroxidase that can be found in various organisms which has the function of reducing and removing hydrogen peroxide and organic peroxide, and protecting cells from oxidative stress. Six isoforms of the PRDX family protein have been identified, which are the thioredoxin-dependent PRDXs 1, 2, 3, 4, 5 and thioredoxin-independent PRDX 6. The PRDX family protein are classified into two subgroups 2-Cys PRDX and 1-Cys PRDX on the basis of the number and the position of the cysteinyl residues involved in catalysis. The PRDX family protein of the present invention includes PRDXs 1, 2, 3, 4, 5 and 6, but is preferably PRDX 5 or PRDX 6.

PRDX 5 is a protein comprising the same or substantially the same amino acid sequence as the amino acid sequence shown by SEQ ID NO:3. It may be a protein derived from any cell or tissue of any organism, preferably an animal, and more preferably a mammal. Examples of mammals include human, chimpanzee, mouse, rat, rabbit, sheep, swine, bovine, horse, cat, dog, and the like, with preference given to human. PRDX 5 may also be a chemically synthesized protein or a protein synthesized using a cell-free translation system. Alternatively, this protein may be a recombinant protein produced from a transformant introduced with a polynucleotide comprising the nucleotide sequence that encodes the above-described amino acid sequence.

As substantially the same amino acid sequence as the amino acid sequence shown by SEQ ID NO:3, an amino acid sequence having a homology of about 80% or more, preferably about 85% or more, more preferably about 90% or more, particularly preferably about 95% or more, and most preferably about 98% or more, to the amino acid sequence shown by SEQ ID NO:3, and the like can be mentioned. As used herein, “homology” means the proportion (%) of the same and similar amino acid residues to all overlapping amino acid residues in the optimal alignment where two amino acid sequences are aligned using mathematic algorithm known in the relevant technical field (preferably, the algorithm is such that a gap can be introduced into one or both of the sequences for the optimal alignment). “A similar amino acid” means an amino acid having similar physicochemical properties; as examples, amino acids classified under the same group, such as aromatic amino acids (Phe, Trp, Tyr), aliphatic amino acids (Ala, Leu, Ile, Val), polar amino acids (Gln, Asn), basic amino acids (Lys, Arg, His), acidic amino acids (Glu, Asp), amino acids having hydroxyl group (Ser, Thr), and amino acids having a small side chain (Gly, Ala, Ser, Thr, Met), can be mentioned. Substitution by such a similar amino acid is expected to produce no change in phenotype of protein (i.e., conservative amino acid substitution). Specific examples of conservative amino acid substitution are known in the relevant technical field and described in various pieces of the literature (see, for example, Bowie et al., Science, 247: 1306-1310 (1990)).

As preferable examples of the protein comprising substantially the same amino acid sequence as the amino acid sequence shown by SEQ ID NO:3, a protein that comprises substantially the same amino acid sequence as the amino acid sequence shown by SEQ ID NO:3 above, and that has substantially the same quality of activity as a protein comprising the amino acid sequence shown by SEQ ID NO:3, and the like, can be mentioned. “Substantially the same quality” means that the proteins are qualitatively (e.g., physiologically or pharmacologically) equivalent to each other. Accordingly, it is preferable that the proteins be equivalent to each other in terms of antioxidant activity (e.g., about 0.01 to 100 times, preferably about 0.1 to 10 times, more preferably 0.5 to 2 times), but quantitative factors such as is the extent of activity and protein molecular weight may be different. Examples of PRDX 5 include human PRDX 5 variants disclosed in GenBank Accession Nos.NP_{—}036226.1 and NP_{—}857635.1.

PRDX 5 can be produced by cultivating a transformant comprising DNA that encodes the protein, and separating and purifying the protein from the culture obtained by a method known per se of protein purification. Specifically, PRDX 5 can be produced by homogenizing transformed cells, and separating and purifying the soluble fraction and/or nuclear fraction by a chromatography such as reversed-phase chromatography, ion exchange chromatography or affinity chromatography, and the like.

Another preferable PRDX family protein, PRDX 6, is known to catalyze redox reactions without depending on thioredoxins unlike other PRDX family proteins. In addition to peroxidase activity, PRDX 6 also has phospholipase A₂ activity, and protects cells from membrane, DNA, and protein damage mediated by ROS-driven oxidative stress or lipid peroxidation. PRDX 6 is a protein comprising the same or substantially the same amino acid sequence as the amino acid sequence shown by SEQ ID NO:5. It may be a protein derived from any cell or tissue of any organism, preferably an animal, and more preferably a mammal. Examples of mammals include human, chimpanzee, mouse, rat, rabbit, sheep, swine, bovine, horse, cat, dog, and the like, with preference given to human. PRDX 6 may also be a chemically synthesized protein or a protein synthesized using a cell-free translation system. Alternatively, this protein may be a recombinant protein produced from a transformant introduced with a polynucleotide comprising the nucleotide sequence that encodes the above-described amino acid sequence.

As substantially the same amino acid sequence as the amino acid sequence shown by SEQ ID NO:5, an amino acid sequence having a homology of about 80% or more, preferably about 85% or more, more preferably about 90% or more, particularly is preferably about 95% or more, and most preferably about 98% or more, to the amino acid sequence shown by SEQ ID NO:5, and the like can be mentioned. The meaning of “homology” is as explained above.

As preferable examples of the protein comprising substantially the same amino acid sequence as the amino acid sequence shown by SEQ ID NO:5, a protein that comprises substantially the same amino acid sequence as the amino acid sequence shown by SEQ ID NO:5 above, and that has substantially the same quality of activity as a protein comprising the amino acid sequence shown by SEQ ID NO:5, and the like, can be mentioned. “Substantially the same quality” means as described above. PRDX 6 can be produced similarly by methods according to those described for PRDX 5.

The preventive or therapeutic agent of the present invention is effective against retinopathy and/or macular degeneration. Examples of retinopathy include diabetic retinopathy, retinopathy of prematurity, and hypertensive retinopathy, but the preventive or therapeutic agent is particularly effective against diabetic retinopathy.

The PRDX family protein of the present invention can be a fusion protein in which it is fused with a PTD (protein transduction domain), which is known to promote delivery of the fused protein into cells of all tissues throughout the body. PTD is not limited as long as the fusion protein (PTD-PRDX family protein) is translocated across a membrane structure and is delivered, into cells, and can be a domain of, for example, 5-50 amino acids, preferably 7-40 amino acids, and more preferably 9-35 amino acids in length, preferably comprising the basic amino acid residues arginine and/or lysine. Examples of PTD include HIV derived TAT domain (SEQ ID NO:1) and Rev domain, Herpes simplex virus derived VP22 domain, and Drosophila derived antennapedia protein, and the like, with preference given to a TAT domain.

PTD can be fused with the N-terminus, C-terminus, or both termini of the PRDX family protein in the PTD-PRDX fusion protein. When PTD is fused with both termini, the PTD may be the same or it may be different. The fusion protein can be obtained by any known method used in the field, including for example, producing a transformant having a fusion gene encoding a PRDX family protein and a PTD using a DNA recombination technique, cultivating the transformant, and then isolating and purifying the target protein from the culture using a method known per se.

The present invention also provides an agent for the prophylaxis or treatment of retinopathy or macular degeneration comprising a polynucleotide encoding a PRDX family protein. The polynucleotide can be DNA, RNA or a chimera thereof, and is preferably a DNA. The polynucleotide encoding a PRDX family protein is preferably a polynucleotide encoding PRDX 5 or PRDX 6.

As the polynucleotide that encodes PRDX 5, genomic DNA or cDNA derived from any cell or tissue of any organism, preferably an animal, and more preferably a mammal (for example, human, chimpanzee, mouse, rat, rabbit, sheep, swine, bovine, horse, cat, dog, and the like, and preferably a human), synthetic DNA and the like can be mentioned. The DNA can also be amplified directly by a reverse transcriptase polymerase chain reaction (hereinafter abbreviated as “RT-PCR method”) using a total RNA or mRNA fraction prepared from the above-described cell or tissue.

As examples of the polynucleotide that encodes PRDX 5, DNA comprising the nucleotide sequence shown by SEQ ID NO:2, DNA that comprises a nucleotide sequence hybridizing to the nucleotide sequence shown by SEQ ID NO:2 under highly stringent conditions, and that encodes the aforementioned protein having substantially the same quality of activity (e.g., antioxidant activity and the like) as a protein comprising the amino acid sequence shown by SEQ ID NO:3, and the like can be mentioned.

Examples of the DNA capable of hybridizing to the nucleotide sequence shown by SEQ ID NO:2 under highly stringent conditions include DNA that comprises a nucleotide sequence showing a homology of about 80% or more, preferably about 85% or more, more preferably about 90% or more, and most preferably about 95% or more, to the nucleotide sequence shown by SEQ ID NO:2.

Hybridization can be conducted according to a method known per se or a method based thereon, for example, a method described in Molecular Cloning, 2nd edition (J. Sambrook et al., Cold Spring Harbor Lab. Press, 1989) and the like. When a commercially available library is used, hybridization can be conducted according to the method described in the instruction manual attached thereto. Hybridization can preferably be conducted under highly stringent conditions.

High-stringent conditions refer to, for example, conditions involving a sodium concentration of about 19 to 40 mM, preferably about 19 to 20 mM, and a temperature of about 50 to 70° C., preferably about 60 to 65° C. In particular, a case wherein the sodium concentration is about 19 mM and the temperature is about 65° C. is preferred.

The polynucleotide that encodes PRDX 5 is preferably DNA comprising the nucleotide sequence shown by SEQ ID NO:2 and the like.

As the polynucleotide that encodes PRDX 6, genomic DNA or cDNA derived from any cell or tissue of any organism, preferably an animal, and more preferably a mammal (as exemplified above), synthetic DNA and the like can be mentioned. The DNA can also be amplified directly by a reverse transcriptase polymerase chain reaction (hereinafter abbreviated as “RT-PCR method”) using a total RNA or mRNA fraction prepared from the above-described cell or tissue.

As examples of the polynucleotide that encodes PRDX 6, DNA comprising the nucleotide sequence shown by SEQ ID NO:4, DNA that comprises a nucleotide sequence hybridizing to the nucleotide sequence shown by SEQ ID NO:4 under highly stringent conditions, and that encodes the aforementioned protein having substantially the same quality of activity (e.g., antioxidant activity and the like) as a protein comprising the amino acid sequence shown by SEQ ID NO:5, and the like can be mentioned.

As examples of the DNA capable of hybridizing to the nucleotide sequence shown by SEQ ID NO:4 under highly stringent conditions, DNA that comprises a nucleotide sequence showing a homology of about 80% or more, preferably about 85% or more, more preferably about 90% or more, and most preferably about 95% or more, to the nucleotide sequence shown by SEQ ID NO:4, and the like are used. Hybridization can be conducted according to the method described above.

The polynucleotide that encodes PRDX 6 is preferably DNA comprising the nucleotide sequence shown by SEQ ID NO:4 and the like.

The polynucleotide of the present invention can be a polynucleotide that encodes a fusion protein comprising a PRDX family protein and a PTD. The nucleotide sequence encoding the PTD is not particularly limited, and includes for example, polynucleotide sequences encoding the above-mentioned TAT domain, Rev domain, VP2 domain or antennapedia protein. The nucleotide sequence encoding a PTD is preferably a nucleotide sequence encoding a TAT domain, and can be exemplified by a nucleotide sequence which is the same or substantially the same as the nucleotide sequence shown by SEQ ID NO:6. A nucleotide sequence which is substantially the same as the nucleotide sequence shown by SEQ ID NO:6 is a nucleotide sequence in which one or more nucleotides are deleted, substituted, inserted, or added to the nucleotide sequence of SEQ ID NO:6. The number of nucleotides that are deleted, substituted, inserted, or added is not particularly limited as long as the protein translocating activity promoted by the PTD encoded by the nucleotide sequence is not lost, but may be for example, from 1 to about 15 nucleotides, preferably from 1 to about 8 nucleotides, more preferably from 1 to about 5 nucleotides, and most preferably 3, 2, or 1 nucleotides.

The polynucleotide of the present invention can be obtained by synthesizing the full length nucleotide sequence by methods known per se, such as by using a commercially available DNA/RNA synthesizer (Applied Biosystems, Beckman, etc.), or isolating the polynucleotide encoding the PRDX family protein from any cell or tissue expressing the same.

The present invention also provides an expression vector in which the above-described polynucleotide operably linked to a promoter has been inserted thereinto. By “operably linked to a promoter”, is meant that the polynucleotide is linked to the promoter so that the promoter allows the polynucleotide to be transcribed.

The backbone of the expression vector of the present invention includes viral vectors and plasmid vectors, preferably viral vectors, but is without limitation as long as the polypeptide of the present invention is expressed in a given host. Examples of viral vectors include adenoviral, retroviral, lentiviral, adeno-associated viral, herpes viral, vaccinia viral, pox viral, polioviral, Sindbis viral, and Sendai viral vectors.

The promoter may be any promoter that can function in a given cell into which the polynucleotide of the present invention is to be introduced, and include viral promoters such as SRα promoter, SV40 early promoter, CMV immediate early promoter, RSV promoter, and MoMuLV promoter, as well as mammalian constitutive promoters such as β-actin promoter, PGK promoter, and transferrin promoter.

The expression vector of the present invention may further comprise elements such as sites for initiation or termination of transcription, ribosome binding site in the transcription region necessary for translation, posttranscriptional regulatory elements such as WPRE, polyadenylation sequences, replication origin, and selectable marker genes such as drug-resistant genes.

The agent of the present invention for preventing or treating retinopathy or AMD, which comprises an effective amount of a PRDX family protein or a polynucleotide encoding the same may be administered to a subject in need thereof.

In a preferred embodiment, the subject is a mammal, preferably a human individual suffering from retinopathy or AMD. A PRDX family protein or a polynucleotide encoding the same is mixed with a pharmacologically acceptable carrier required to yield an agent, and then administered to the subject.

Here, as examples of the pharmacologically acceptable carrier, various organic or inorganic carrier substances conventionally used as pharmaceutical preparation materials can be mentioned, and these are formulated as excipients, lubricants, binders and disintegrants, in solid preparations; as solvents, solubilizing agents, suspending agents, isotonizing agents, buffers and soothing agents, in liquid preparations, and the like. Also, as necessary, pharmaceutical preparation additives such as antiseptics, antioxidants, coloring agents and the like can be used.

As examples of suitable excipients, lactose, saccharose, D-mannitol, D-sorbitol, starch, gelatinized starch, dextrin, crystalline cellulose, low substituted hydroxypropyl cellulose, sodium carboxymethyl cellulose, gum arabic, pullulan, light silicic anhydride, synthetic aluminum silicate, magnesium metasilicate aluminate and the like can be mentioned.

As examples of suitable lubricants, magnesium stearate, calcium stearate, talc, colloidal silica and the like can be mentioned.

As examples of suitable binders, gelatinized starch, sucrose, gelatin, gum arabic, methyl cellulose, carboxymethyl cellulose, sodium carboxymethyl cellulose, crystalline cellulose, saccharose, D-mannitol, trehalose, dextrin, pullulan, hydroxypropyl cellulose, hydroxypropylmethyl cellulose, polyvinyl pyrrolidone and the like can be mentioned.

As examples of suitable disintegrants, lactose, saccharose, starch, carboxymethyl cellulose, calcium carboxymethyl cellulose, sodium crosscarmellose, sodium carboxymethyl starch, light silicic anhydride, low substituted hydroxypropyl cellulose and the like can be mentioned.

As examples of suitable solvents, water for injection, physiological saline, Ringer's solutions, alcohols, propylene glycol, polyethylene glycol, sesame oil, corn oil, olive oil, cottonseed oil and the like can be mentioned.

As examples of suitable solubilizing agents, polyethylene glycol, propylene glycol, D-mannitol, trehalose, benzyl benzoate, ethanol, trisaminomethane, cholesterol, triethanolamine, sodium carbonate, sodium citrate, sodium salicylate, sodium acetate and the like can be mentioned.

As examples of suitable suspending agents, surfactants such as stearyl triethanolamine, sodium lauryl sulfate, lauryl aminopropionic acid, lecithin, benzalkonium chloride, benzethonium chloride and glyceryl monostearate; hydrophilic polymers such as polyvinyl alcohol, polyvinyl pyrrolidone, sodium carboxymethyl cellulose, methyl cellulose, hydroxymethyl cellulose, hydroxyethyl cellulose and hydroxypropyl cellulose; polysorbates, polyoxyethylene hardened castor oil and the like can be mentioned.

As examples of suitable isotonizing agents, sodium chloride, glycerin, D-mannitol, D-sorbitol, glucose and the like can be mentioned.

As examples of suitable buffers, buffer solutions of a phosphate, an acetate, a carbonate, a citrate and the like, and the like can be mentioned.

As examples of suitable soothing agents, benzyl alcohol and the like can be mentioned.

As examples of suitable antiseptics, paraoxybenzoates, chlorobutanol, benzyl alcohol, phenethyl alcohol, dehydroacetic acid, sorbic acid and the like can be mentioned.

As examples of suitable antioxidants, sulfides, ascorbates and the like can be mentioned.

As examples of suitable coloring agents, water-soluble food tar colors (e.g., food colors such as Food Red Nos. 2 and 3, Food Yellow Nos. 4 and 5, and Food Blue Nos. 1 and 2), water-insoluble lake pigments (e.g., aluminum salts of the aforementioned water-soluble food tar colors and the like), natural pigments (e.g., β-carotene, chlorophyll, red iron oxide and the like) and the like can be mentioned.

The agent of the present invention can be administered orally or non-orally, as is appropriate for the subject. Examples of dosage forms of the aforementioned agent include oral formulations such as tablets, capsules (including soft capsules and microcapsules), granules, powders, syrups, emulsions and suspensions; and non-oral formulations such as eye drops, ophthalmic ointments, injections (e.g., subcutaneous injections, intravenous injections, intramuscular injections, intraperitoneal injections and the like), external formulations (e.g., nasal preparations, transdermal preparations and the like), pellets, sustained-release preparations (e.g., sustained-release microcapsules and the like) and the like.

The agent can be produced by a method conventionally used in the field of pharmaceutical preparation making, for example, a method described in the Japanese Pharmacopoeia and the like. The content of the protein or the polynucleotide of the present invention in the agent varies depending on the dosage form, the dose of the compound and the like; and is, for example, from about 0.001 to 100% by weight.

When the dosage form of the agent of the present invention is an injection or an eye drop, it can be produced by dissolving, suspending or emulsifying the active ingredient in an aqueous solvent (e.g., distilled water, physiological saline, Ringer's solution and the like), an oily solvent (e.g., vegetable oils such as olive oil, sesame oil, cottonseed oil and corn oil, propylene glycol, and the like), or the like, along with a dispersing agent (e.g., polysorbate 80, polyoxyethylene hydrogenated castor oil 60, polyethylene glycol, carboxymethyl cellulose, sodium alginate and the like), a preservative (e.g., methylparaben, propylparaben, benzyl alcohol, chlorobutanol, phenol and the like), an isotonizing agent (e.g., sodium chloride, glycerin, D-mannitol, D-sorbitol, glucose and the like), and the like. At this time, if desired, additives such as a solubilizing agent (e.g., sodium salicylate, sodium acetate and the like), a stabilizer (e.g., human serum albumin and the like), a soothing agent (e.g., benzyl alcohol and the like) and the like may also be used.

The dosage of the protein or the polynucleotide of the present invention varies depending on the subject of administration, dosage form and the like; in an adult human patient infected with retinopathy or AMD (body weight 60 kg), for example, the dosage is about 0.1 to 1000 mg, preferably about 1.0 to 100 mg, more preferably about 10 to 50 mg, per day.

The present invention is hereinafter described in more detail by means of the following examples, which, however, are not to be construed as limiting the present invention.

### Experimental Procedures

### (1) Animals and Culture

All animal experiments were accepted by the committee of animal research in University of Fukui, Japan and conducted in accordance with the National Institute of Health Guide for the Care and Use of Laboratory Animals and the animal protocol in University of Fukui.

4 and 7-week-old, Sprague-Dawley albino rats were used, which were obtained from a local animal dealer (Clea Japan Inc., Osaka, Japan). To measure the expressions of PRDXs 1-6, 7-week-old, female rats were used for the real-time PCR (n=8), protein blot (n=8) and the immunohistochemistry (n=6).

Diabetic rats were induced in female-, 4-week-old rats (n=8) by a single intraperitoneal injection of 80 mg/kg body weight streptozotocin (STZ) (Sigma, St. Louis, Mo., USA) in 0.05 mM citrate buffer (pH 4.5) after they had been fasted overnight (Rakieten N et al., 1963, Cancer Chemother Rep. 29, 91-98). Female, 4-week-old rats (n=8) were used as normal control animal. These control and STZ-injected rats were given ad libitum access to regular chow consisting of 25% (w/w) protein, 53% carbohydrate, 6% fat, and 8% water (Oriental Yeast Co. Ltd., Osaka, Japan) for 31 weeks. In the 4th week after STZ injection, all rats had blood glucose levels >600 mg/ml (33.4 mmol/l). At 31 weeks after STZ injection, these 35-week-old rats were used for experiments as STZ rats. Other 35-week-old rats were used as control.

Primary cultures of retinal capillary pericytes were isolated from pig retinal microvessels as described previously (Gitlin and D'Amore, 1983, Microvasc Res. 26, 74-80). Briefly, porcine eyes were obtained from the local abattoir and retinas isolated and homogenized in Dulbecco's Modified Eagle's medium (DMEM) (Invitrogen, Carlsbad, Calif.). The homogenate was filtered through a 100 μm Nylon cell strainer (Beckton Dickinson, Bedford, Mass.). The trapped microvessels were digested in the collagenase and deoxyribonuclease (Sigma) in phosphate-buffered saline (PBS; pH7.4) for 20 minutes at 37° C., filtered is through a 70 μm Nylon cell strainer (Beckton Dickinson), and plated in 25 cm² tissue culture flasks (Nalge Nunc, Rochester, N.Y.). Cells were cultured in DMEM supplemented with 15% fetal bovine serum (FBS) (Sigma, St. Louis, Mo.) and 1% antibiotic-antimycotic (penicillin G sodium, streptomycin sulfate, and amphotericin B) (Sigma). The retinal pericytes in culture were identified by positive staining for α-SMA antigen and negative staining for factor VIII-related antigen (Sigma). Cells generated after 3 to 6 such passages were used in the experiments.

Pericytes were cultured in DMEM supplemented with 15% FBS and antibiotics (100 μg/ml streptomycin, 100 U/ml penicillin) at 37° C. in 6% CO₂. To monitor the expression of PRDXs 5 and 6 after high glucose culture, pig pericytes were cultured with DMEM containing 5.5 mM (5 G) or 30 or 50 mM (30 G or 50 G) D-glucose (Sigma) supplemented with 5% FBS medium. For osmotic control, pig pericytes were grown in 5.5 mM D-glucose medium containing 24.5 or 44.5 mM (30M or 50M) mannitol (Sigma). For transduction of TAT-HA-PRDXs 5 and 6, cells were grown overnight on 6-well plates, and then 10 μg/mL of TAT-HA-PRDX 5 and 6 proteins were added to the culture media. After incubation periods of 24 hr, cells were washed and incubated further for 0 or 24 hr and harvested for the preparation of cell extracts. Three experiments were done for each assay using 4 different isolates.

### (2) Prokaryotic Expression of PRDXs 5 and 6

The cDNA encoding the open reading frame of PRDX 5 or 6 was isolated from human LEC cDNA library (Fatma et al., 2001, supra) using PRDX 6 specific sense (5′GTCGCCATGGCCGGAGGTCTGCTTC-3′ (SEQ ID NO:7) contained Ncol site) and antisense primer (5′AATTGGCAGCTGACATCCTCTGGCTC-3′ (SEQ ID NO:8)), and PRDX 5 specific sense (5′GCTGGTACCATGGCCCCAATCAAGGTGGGA-3′ (SEQ ID NO:9) contained kpnI site) and antisense primer (5′TAGAATTCAGAGCTGTGAGATGA TATTGG-3′ (SEQ ID NO:10)). The cDNA encoding the open reading frame of PRDX 5 or 6 was subcloned in-frame downstream of the N-terminal 6xHis-TAT protein transduction domain sequences in the pTAT-HA (hemaglutinin-tagged) expression vector as described in the present inventors' previous study (Kubo et al., 2008, supra). For the preparation of TAT-HA-PRDX 6 constructs, the present inventors followed the method of Dowdy et al. (Schwarze and Dowdy, 2000, Trends Pharmacol Sci. 21, 45-48; Vocero-Akbani et al., 2000, Methods Enzymol. 322, 508-521.). Expression and purification of TAT-HA-PRDX 5 and 6 fusion proteins were followed as described in the present inventors' previous study (Kubo et al., 2008, supra).

### (3) Real-Time RT-PCR

To monitor the levels of PRDXs 1-6 in rat retina, total RNA was isolated from 6 rats using the single-step guanidine thiocyanate/phenol/chloroform extraction method (Trizol Reagent, Invitrogen) and converted to cDNA using Ready-To-Go You-Prime First-Strand Beads (Amersham Biosciences, Piscataway, N.J.). To monitor the levels of PRDXs 5 and 6 in pig pericytes, total RNA was isolated using RNeasy® Mini Kit (Qiagen Inc., Turnberry Lane, Valencia) and converted to cDNA using Ready-To-Go You-Prime First-Strand Beads (Amersham). To validate the expression patterns of pig PRDXs 5 and 6, relative quantification of mRNA was performed using Prism 7000 (Applied Biosystems, Foster City, Calif.). PCR amplification was performed with TaqMan Universal Master Mix (Applied Biosystems). The relative quantities of rat or pig PRDX 1-6 mRNA were obtained using the comparative Ct method and was normalized using pre-developed TaqMan assay reagent human ribosomal RNA as an endogenous control (Applied Biosystems).

In each group, the Cts of target genes were normalized to the levels of ribosomal RNA used as an endogenous control. The ΔCt for each gene was calculated as described previously (Kubo et al., 2005, supra). Three experiments were done for the each assay using 4 different isolates in each group.

### (4) Western Blot Analysis

Protein lysates of rat retinal tissue or pig retinal pericytes were prepared in ice-cold radioimmune precipitation buffer as described previously (Kubo et al., 2003, Histochem Cell Biol. 119, 289-299). Twenty micrograms of protein were loaded and run on a 10-20% SDS-PAGE gradient gel and transferred to a PVDF membrane (BioRad Laboratories, Hercules, Calif.). The membranes were blocked with 5% milk and were incubated overnight at 4° C. with anti-PRDX 1-6 monoclonal Ab (LabFrontier, Seoul, Korea) (dilution 1:3000). After being washed, the membranes were incubated with anti-mouse IgG labeled with horseradish peroxidase (diluted 1:2000; Santa Cruz Biotechnology Inc., Santa Cruz, Calif.) and visualized by the enhanced chemiluminescence method according to the manufacturer's protocol (Santa Cruz Biotechnology). The same dilutions of absorbed Abs to the recombinant PRDX 5 and 6 proteins were used as negative controls. Anti-rabbit β-actin Ab (Sigma) was used to show that equal amount of protein was loaded in each lane. The present inventors used LAS-3000mini (Fujifilm, Tokyo, Japan), which is an image analysis system dedicated to chemiluminescence applications in all experiments for western blot. Densities (pixels) of captured images were analyzed using Science Lab software (Fujifilm) and Multigauge software (Fujifilm) and relative densities to control samples were calculated using Multigauge software (Fujifilm). Three independent experiments were performed in the each protein blot assay using four different isolates in each group.

### (5) Immunohistochemical Localization of PRDXs 5 and 6 in Rat Tetina

The 4 rats' eyes were fixed in 4% paraformaldehyde in phosphate buffered saline and embedded in paraffin and sectioned at 4 μm.

Immunostaining was performed using the Tyramide Signal Amplification (TSA™) Kit (Molecular Probes Inc., Eugene, Oreg.), following the manufacturer's protocol. The tissue sections were exposed to the anti-PRDX 5 or 6 monoclonal Ab (LabFrontier) (dilution 1:2000) or a-smooth muscle actin (α-SMA) Ab (dilution 1:500) (Sigma) overnight, followed by incubation in horseradish peroxidase-conjugated goat anti-rabbit IgG (Molecular Probes) diluted to 1:100. Tyramide working solution was applied to the specimens. The same dilutions of absorbed Abs to the recombinant PRDX 5 and 6 proteins were used as negative controls. Preparation of HA recombinant PRDX 6 protein has been reported elsewhere (Fatma et al., 2001, supra; Kubo et al., 2008, supra). Staining of nuclei was performed using Hoechst solution (Molecular Probes) following immunostainings.

(6) Cell Viability Assays and TdT-Mediated dUTP-Biotin Nick End Labeling (TUNEL)

For cell survival assays, pig pericytes were cultured with 5.5, 30 or 50 mM D-glucose (5 G, 30 G or 50 G) for 2 days and then cultured with 5.5, 30 or 50 mM D-glucose (5 G, 30 G or 50 G) in the presence of 10 μg/ml of PRDX 5 or bovine serum albumin for 4 (Day 6) and 8 (Day 10) days. Then, cell proliferation assay using 3-(4,5-dimethylthiazol-2-yl)-5-(3-carboxymethoxyphenyl)-2-(4-sulfophenyl)-2H-tetrazolium, inner salt (MTS) (Promega, Madison, Wis.) was performed to monitor the number of surviving cells in each group. Absorbance of pig pericytes cultured with 5.5 mM D-glucose was measured as 100% cell survival, and the percentage cell survival was then calculated for each group. Three experiments were done for each assay using 4 different isolates in each group.

The TUNEL assay was performed to assess apoptotic cell death. Pig pericytes were cultured for 6 and 10 days with DMEM/5% FBS medium containing 5.5 mM (5 G), 30 mM (30 G) or 50 mM D-glucose (50 G) treated with/without 10 μg/mL TAT-HA-PRDX 5 or 6 recombinant protein in 4-well chamber slides (Nalge Nunc International Corp., Naperville, Ill.) and then subjected to TUNEL staining (ApoAlert DNA fragmentation assay, BD Bioscience) and staining with 4′,6-diamidino-2-phenylindole dihydrochloride (DAPI) (Dojindo Laboratories, Kumamoto, Japan). Four experiments were done for each assay using different isolates. The total number of cells and the number of apoptotic cells in each group were estimated. The percentage of apoptotic cells (TUNEL-positive cells) per total number of cells was counted from the number of DAPI-stained nuclei in six different fields of each slide for each group using the Scion Image software. Three experiments have done for each assay using 4 different isolates in each group.

In the present inventors' preliminary unpublished data and previous study (Kubo et al., 2008, supra), 2.5-20 μg/mL TAT-HA-PRDX 5 or 6 recombinant proteins were effective in reducing ROS level in human cultured LECs. Following these data, the present inventors used 10 μg/mL TAT-HA-PRDX 5 or 6 recombinant protein in this study.

(7) Detection of Oxidative Stress-Induced DNA Damage using 8-hydroxy-2′-deoxyguanosine (8-OHdG) immunohistochemistry

Pig pericytes were cultured for 10 days with DMEM/5% FBS medium containing 5.5 mM (5 G) or 30 mM D-glucose (30 G) treated with/without 10 μg/mL TAT-HA-PRDX 5 and 6 recombinant proteins in 4-well chamber slides (Nalge Nunc) and were then subjected to anti-8-OHdG immunostaining. Four experiments were done for each assay using different isolates. Immunostaining was carried out using a DAKO LSAB Kit for rabbit and mouse primary Abs (DAKO, Carpinteria, Calif.) according to the company's protocol. They were then exposed overnight to the anti-8OHdG monoclonal Ab (JaIKA, Nikken SEIL Co., Ltd, Shizuoka, Japan) (dilution 1:500). The Ab complex was visualized by adding a 0.02% (v/v) solution of 3,3-diaminobenzidine (Bio-Rad). Three experiments were done for each assay using 4 different isolates in each group.

### (8) Statistical Analysis

The results are reported as means±standard deviation and were analyzed statistically using ANOVA with Fisher's test.

### EXAMPLE 1

### PRDXs 5 and 6 are Highly Expressed in Rat Retina

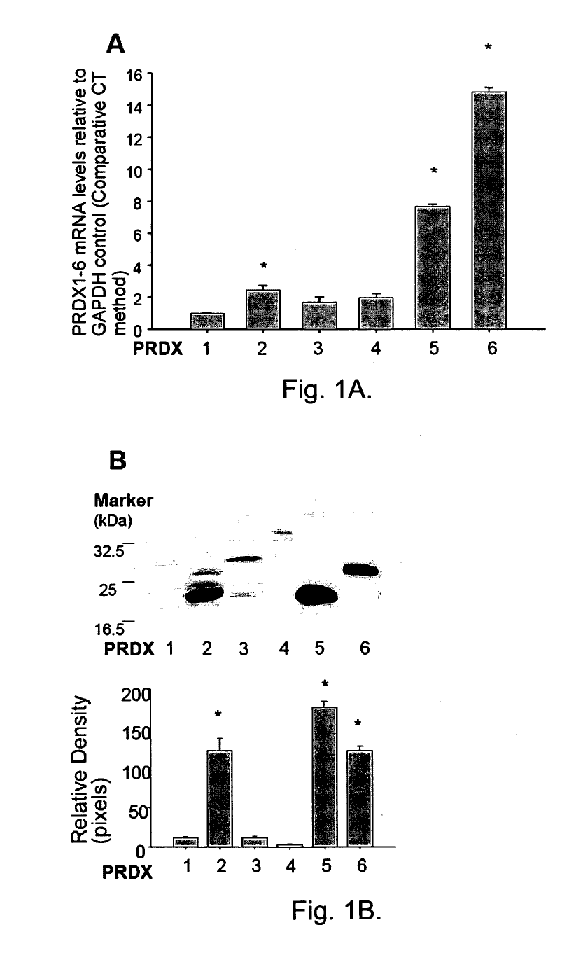

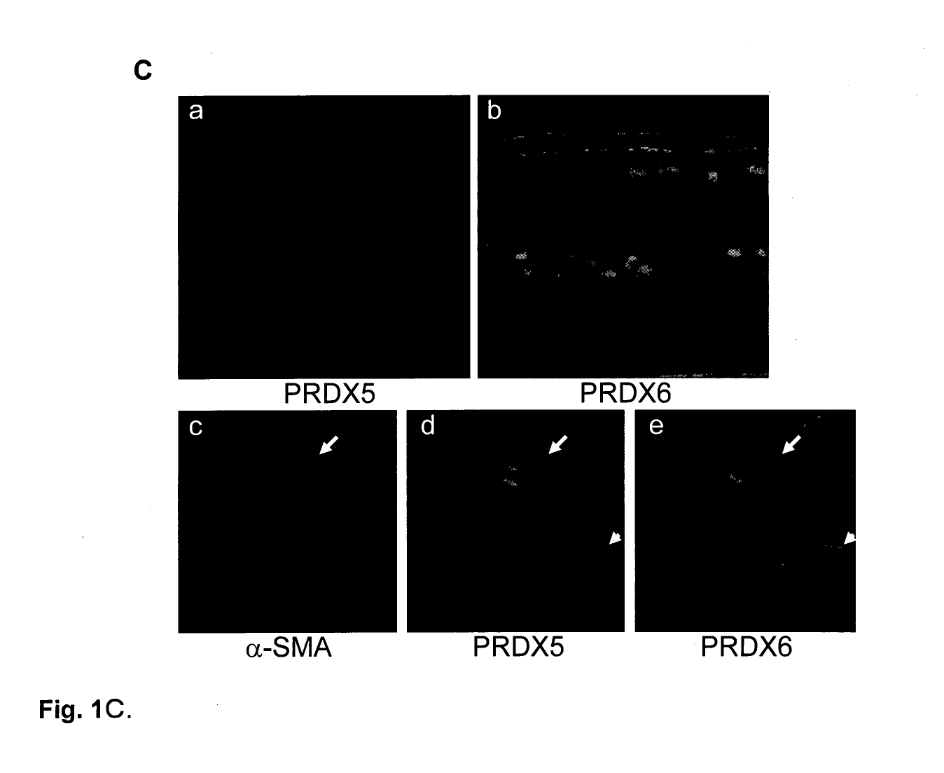

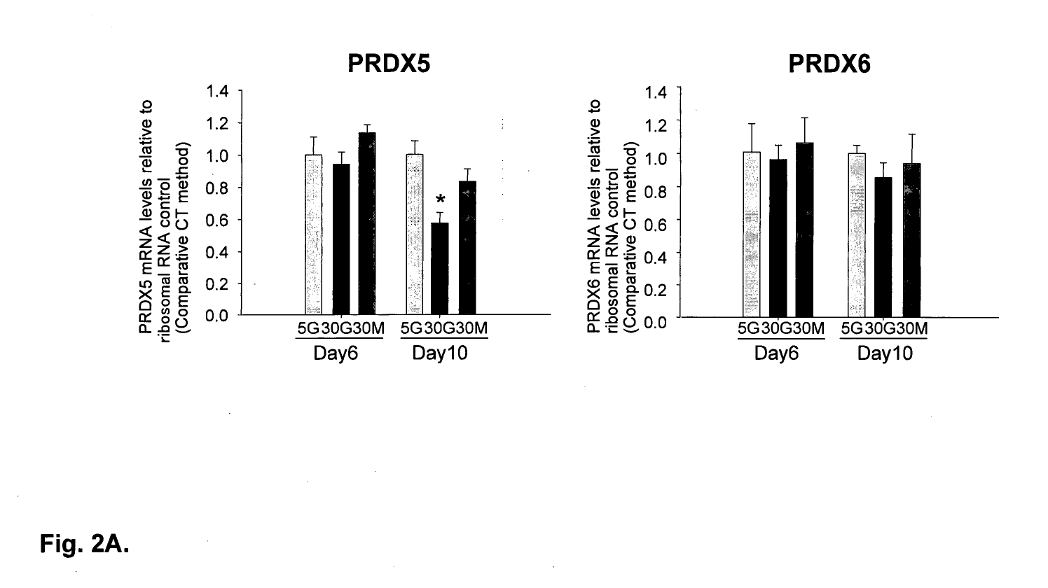

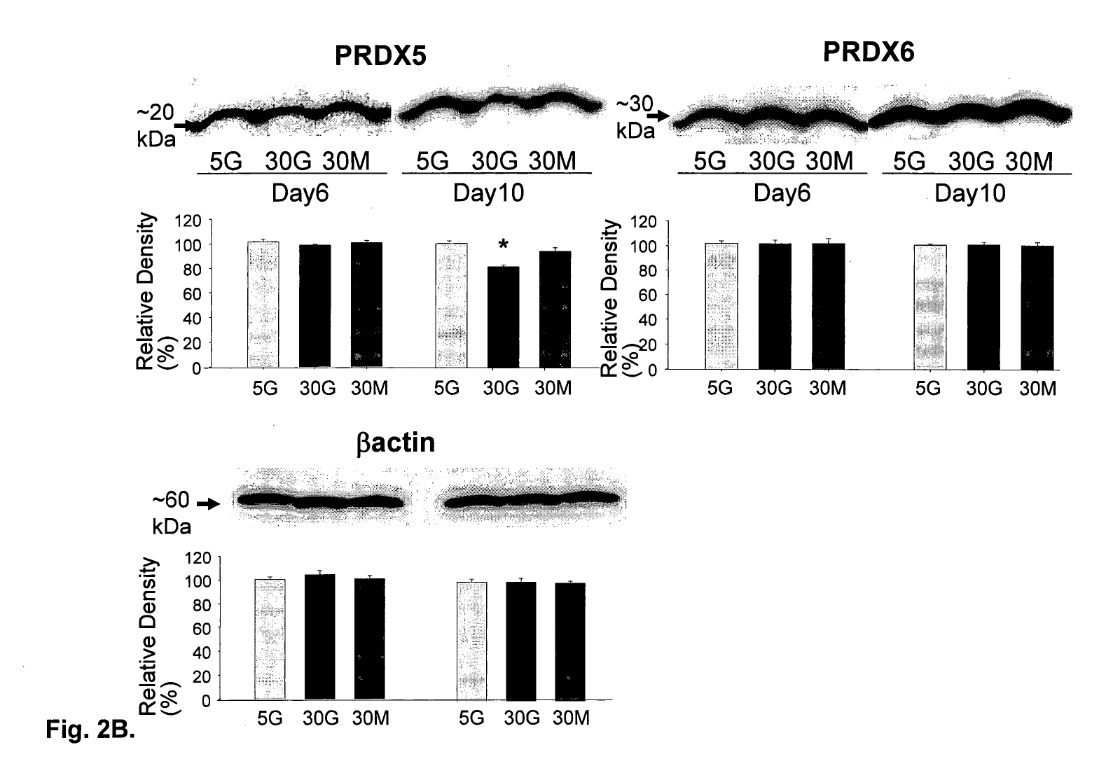

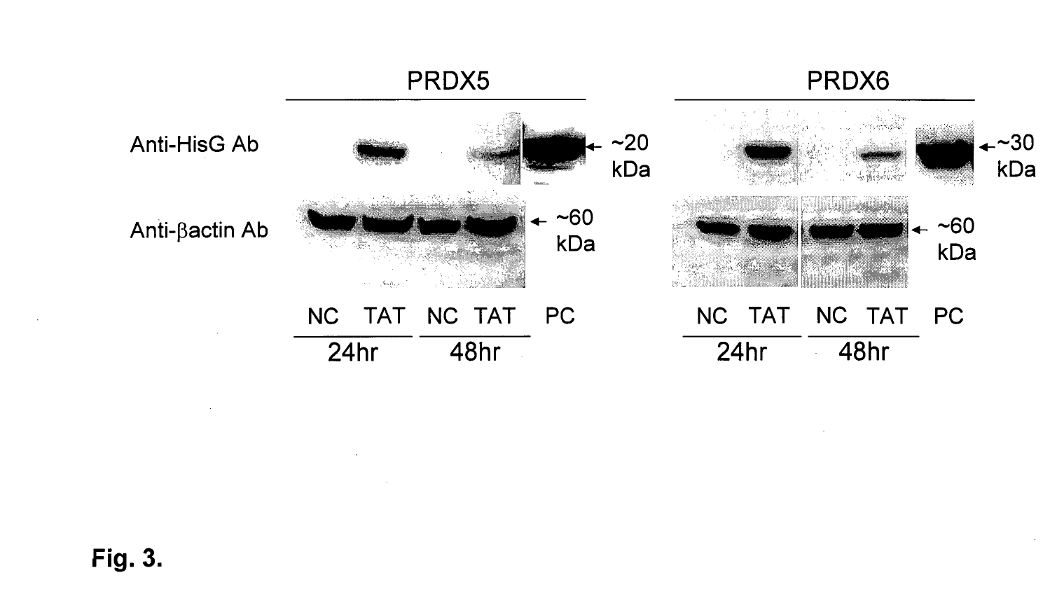

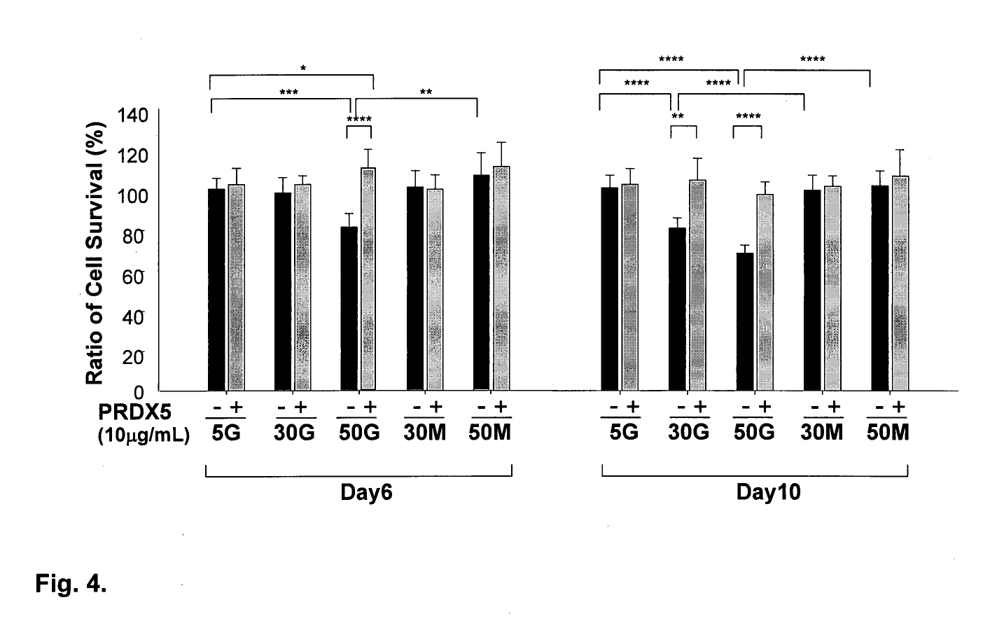

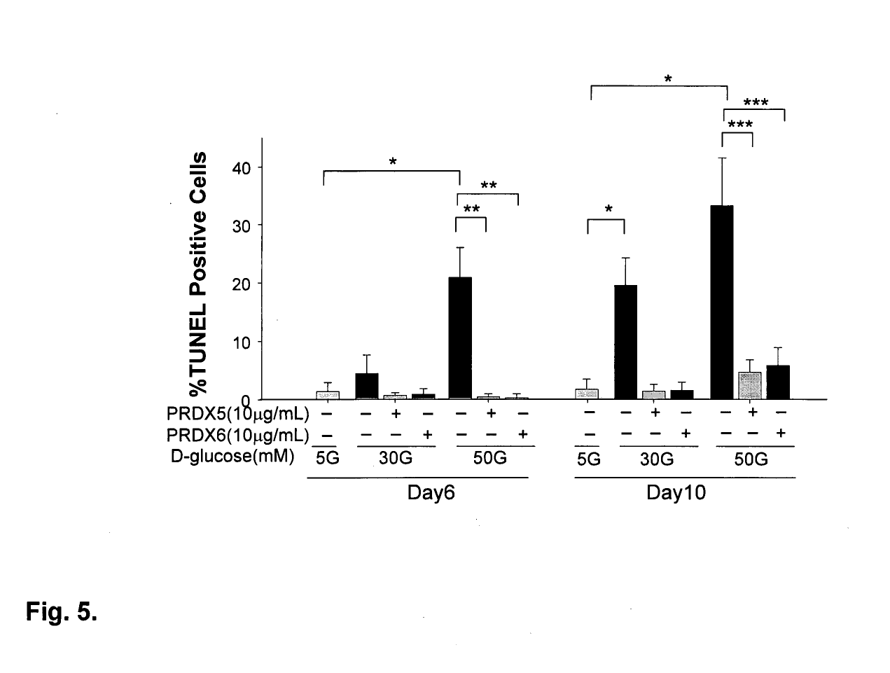

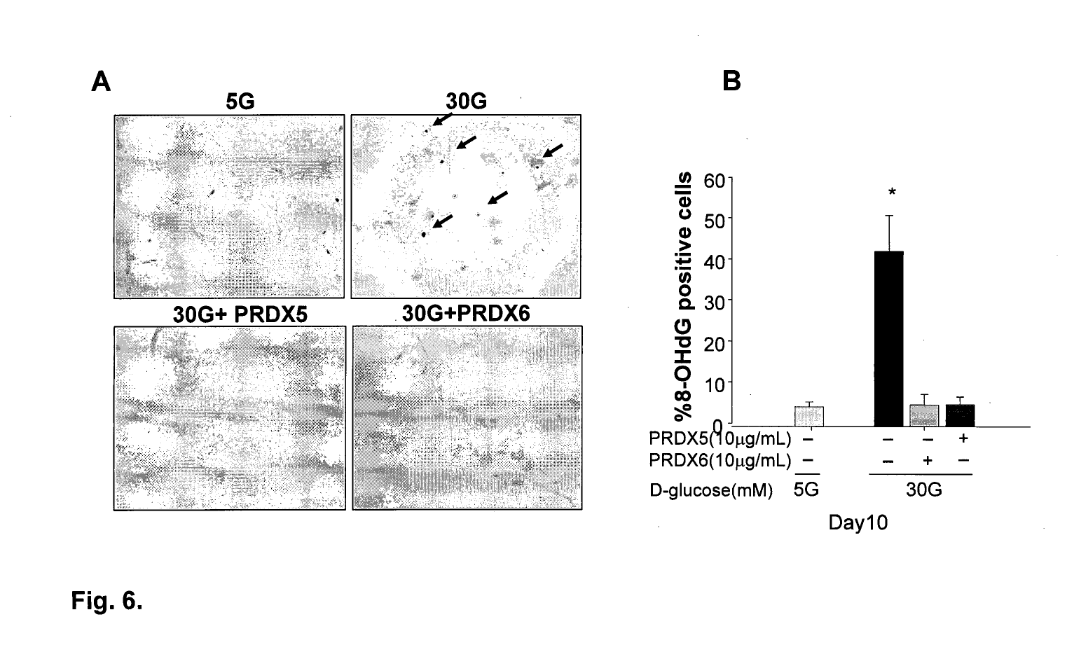

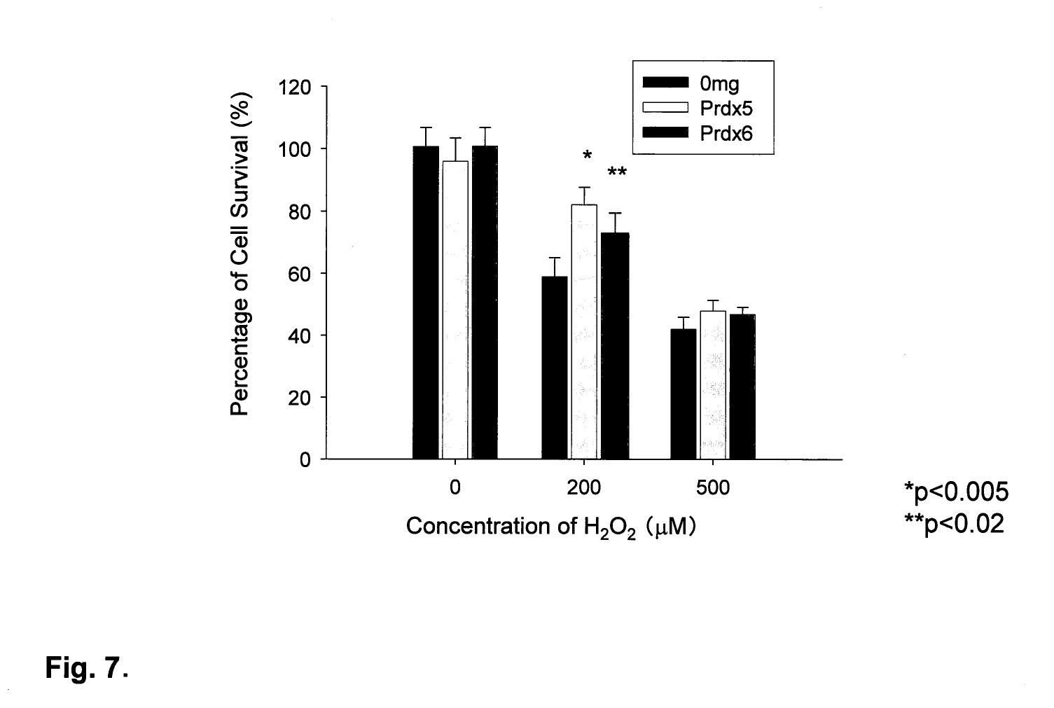

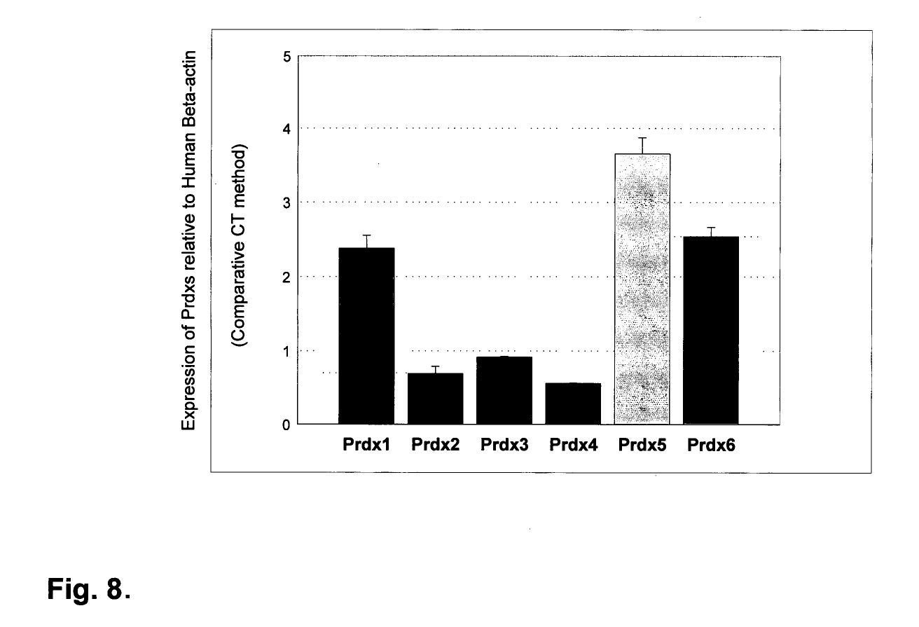

According to a recent classification, PRDX family consists of six members (PRDXs 1-6). An initial examination of all the members using real-time RT-PCR and western blot analysis revealed higher expressions of PRDXs 5 and 6 in the rat retina (FIGS. 1A and B) than other PRDX members (PRDXs 1-4). On the basis of these results, the present inventors selected PRDXs 5 and 6 for the following study using pig pericytes.

Immunohistochemistry was performed to analyze the expressions and localizations of PRDXs 5 and 6 in the sections of 7 week-old rat eyes. PRDXs 5 and 6 were expressed in the cytoplasm of whole layers of retina (FIG. 1C-a and -b, respectively). α-SMA expresses in pericyte of rat retinal vessels (FIG. 1C-c). PRDXs 5 and 6 were co-localized with α-SMA in pericytes of rat retina (FIG. 1C-d and -e, respectively). These results implicate that PRDXs 5 and 6 were expressed in rat pericytes.

To examine the effect of hyperglycemia on the expressions of PRDXs 5 and 6, the present inventors measured the levels of PRDX 5 and 6 proteins in 35-week-old STZ and age-matched control rats using real time PCR and protein blot methods. Levels of PRDX 5 and 6 mRNA and protein expressions did not significantly alter in whole retina of 35 weeks-old STZ rats comparing with control 35 weeks-old rats.

### EXAMPLE 2

### Expression of PRDXs 5 and 6 in High-Glucose-Exposed Pig Pericytes

The present inventors have previously shown that PRDX 6 expression is reduced in high-glucose-exposed human LECs in comparison to normal (5 mM)-glucose exposed LECs, leaving them vulnerable to oxidative stress (Kubo et al., 2004, supra). Therefore, the present inventors investigated the expression levels of PRDX 5 and 6 proteins in high-glucose-exposed pig pericytes. Expressions of PRDX 5 mRNA and protein were significantly down-regulated in pig pericytes cultured with high-glucose medium for 10 days compared to those cultured with normal glucose medium (5 G) (FIGS. 2A and B). In contrast, PRDX 6 mRNA level was not decreased in pig pericytes cultured in high-(30 G) and normal (5 G) glucose media for 10 days (FIGS. 2A and B).

### EXAMPLE 3

TAT-HA-PRDX 5 and 6 Fusion Proteins were Able to Enter Cultured Pig Pericyte

Western blot analysis revealed that TAT-HA-PRDXs 5 and 6 were transduced into the cells after 24 and 48 hr (FIG. 3; TAT). The present inventors could not detect any band when HA-PRDX 6 with flag tag (HA) only was added to the culture medium as a control (FIG. 3; NC).

### EXAMPLE 4

### TAT-HA-PRDX 5 Protected Pig Pericyte Against High-Glucose Induced Cell Death

A cell viability assay (MTS assay) revealed that cell survival gradually decreased in pig pericytes cultured with 30 mM D-glucose (30 G) for 10 days and 50 mM D-glucose (50 G) for 6 and 10 days (FIG. 4). Addition of PRDX 5 significantly blocked the increased inhibitory effect of high glucose (30 G or 50 G) on pericytes at 6 and 10 days after culture. A hyperosmolar condition (30M; 5.5 mM glucose+24.5 mM mannitol, 50M; 5.5 mM glucose+44.5 mM mannitol) had no effect on cell viability (FIG. 4). These results suggest that PRDX 5 prevented high-glucose-induced cell death through its antioxidant property.

Next, the present inventors investigated whether PRDXs 5 and 6 could protect against high-glucose (30 G and 50 G)-induced apoptotic cell death in pericytes using the TUNEL assay. As shown in FIG. 5, PRDXs 5 and 6 significantly inhibited apoptotic cell death in the group cultured with 50 mM D-glucose (50 G) after 6 days and high-glucose (30 G and 50 G)-exposed pericytes after 10 days. These observations suggest that PRDXs 5 and 6 could protect retinal pericytes against high-glucose-induced apoptosis through its antioxidant property.

### EXAMPLE 5

### TAT-HA-PRDXs 5 and 6 Protected Pig Pericytes Against Oxidative Stress-Induced DNA Damage in High- and Normal Glucose Exposure

The present inventors also investigated whether PRDXs 5 and 6 could protect against oxidative stress-induced DNA damage in pericytes using anti-8-OHdG Ab. 8-OHdG is known as a biomarker of oxidative stress-induced DNA damages (Morita et al., 2005, Curr Neurovasc Res. 2, 113-120; Nishigori et al., 2005, Br J Dermatol. 153 Suppl 2, 52-56; Sato et al., 2005, Neurology. 64, 1081-1083; Tarng et al., 2000, Am J Kidney Dis. 36, 934-944), because deoxyguanosine (dG) is one of the constituents of DNA, and when oxidized, it is converted into 8-OHdG. Ten days after high-glucose (30 G) culture, 8-OHdG positive-pericytes were significantly increased in high-glucose (30 G)-exposed pericytes, however, PRDXs 5 and 6 significantly inhibited oxidative stress-induced DNA damage in high glucose culture (FIG. 6).

### EXAMPLE 6

### TAT-HA-PRDXs 5 and 6 Protected RPE Against H₂O₂ Induced Cell Death

Adult human retinal pigment epithelial cells (ARPE19) were purchased from the American Tissue Culture Collection (Bethesda, USA). These cells were maintained in Dulbecco's Modified Eagle's Medium/Nutrient Mixture F-12 (DMEM/F12) supplemented with 10% fetal calf serum (Invitrogen, Carlsbad, Calif.), 100 units/ml penicillin, and 100 μg/ml streptomycin (Sigma). The cells were cultured in a humidified incubator at 37° C. and 5% CO₂. Cells reaching a 90%-95% of confluence were starved and synchronized in serum-free DMEM for 8 hr before they were subjected to further analysis.

Cultured human RPE were incubated with different concentrations of recombinant PRDX 5 and 6 proteins linked to the Trans-Activating Transduction (TAT) domain from HIV-1 (TAT-HA-PRDXs 5 and 6) for 24 hours. Cells were then treated with 200 to 500 μM hydrogen peroxide (H₂O₂) for 2 hours. Cell survival was measured by using MTS assay. H₂O₂ treatment caused significant decreases in cell vitality (40-60%) of human RPE. TAT-HA-PRDX 5 and PRDX 6 significantly inhibited H₂O₂-induced RPE cell death as shown in FIG. 7. Addition of TAT-HA-PRDX 5 was more effective on H₂O₂-induced RPE cell death than TAT-HA-PRDX 6. Results suggest that delivery of PRDX 5 and PRDX 6 can protect RPE from oxidative stress in human RPE.

The importance of PRDX enzymes is underlined by the high abundance in the cytosol and involvement in multiple cellular processes ranging from antioxidant defenses (Kim et al., 2000, J Biol Chem. 275, 18266-18270; Neumann et al., 2003, Nature. 424, 561-565; Wood et al., 2003a, supra), parasite drug resistance (Sherman et al., 1996, Science. 272, 1641-1643), cancer (Chung et al., 2001, Anticancer Res. 21, 1129-1133; Neumann et al., 2003, supra; Park et al., 2000, Clin Cancer Res. 6, 4915-4920) to H₂O₂-mediated cellular signaling (Choi et al., 2005, Nature. 435, 347-353; Vivancos et al., 2005, Proc Natl Acad Sci USA. 102, 8875-8880), and regulation of cell proliferation. PRDXs are known as stress-response proteins. Expressions of PRDXs are induced by oxidative stress mediated by H₂O₂, glucocorticoids, and ultraviolet irradiation (Fatma et al., 2005, Cell Death Differ. 12, 734-750; Fatma et al., 2001, supra). In mouse lenses and retinal ganglion cells and, the expression of PRDX 6 is highest in PRDXs 1-6 (Fatma et al., 2008, Brain Res. 1233, 63-78; Fatma et al., 2005, supra; Kubo et al., 2006, Mech Ageing Dev. 127, 249-256). In lens, expression of PRDX 5 is higher than PRDXs 1-4, however, in cultured retinal ganglion cells, expression of PRDX 5 was lowest of PRDXs 1-6 (Fatma et al., 2008, supra; Fatma et al., 2005, supra). In the rat retina, gene expressions of PRDX 5 and 6 are higher than those of other PRDX members. Although, PRDX 2 protein is also present at significant level, the present inventors selected PRDXs 5 and 6 in this study because expression of PRDX 2 was lower in comparison to PRDX 5 or 6 in lens and retinal ganglion cells in the present inventors' previous studies (Fatma et al., 2008, supra; Fatma et al., 2005, supra). To study the protective ability of PRDXs in the total diabetic eye complications including diabetic cataract, rubeotic glaucoma and diabetic retinopathy, the present inventors selected PRDXs 5 and 6 in this study.

The present study demonstrates that PRDXs 5 and 6 play an important role in protecting the retinal pericytes from the high glucose induced oxidative damage. The expression of PRDX 5 was decreased in high-glucose cultured pig retinal pericytes. The reduction of PRDX 5 expression may induce the production of ROS in pig pericytes, however, the expression of PRDX 5 was not modified in retinal tissues obtained from STZ-induced diabetic rats. Because of the population of pericytes in retinal cells in retinal tissues, the present inventors could not estimate the expression levels of PRDX 5 in retinal pericytes using the total retina. Further studies may be required using vascular tissues obtained-from the retinal trypsin digests to analyze it. However, it is clear that PRDXs 5 and 6 significantly prevented the high-glucose-induced apoptotic cell death, a predictor of retinopathy (Kern et al., 2000, Invest Ophthalmol Vis Sci. 41, 3972-3978; Mizutani et al., 1996, J Clin Invest. 97, 2883-2890), and the early signs of retinal pathology in diabetic rats. Increased oxidative stress in diabetes is considered a contributing factor in the development of diabetic complications, including retinopathy (Baynes and Thorpe, 1999, Diabetes. 48, 1-9; Haskins et al., 2003, Ann NY Acad Sci. 1005, 43-54; Kowluru et al., 2001, supra). Superoxide levels are elevated (Du et al., 2003, Free Radic Biol Med. 35, 1491-1499), mRNA levels of SOD are downregulated (Li et al., 1999, Cell Mol Biol (Noisy-le-grand). 45, 59-66), and glutathione levels are decreased in diabetic retina or high-glucose induced rat or pig pericyte (Kowluru, 2003, supra; Manea et al., 2004, J Cell Mol Med. 8, 117-126; Sharpe et al., 1998, Diabetes. 47, 801-809) suggesting overwhelming of the endogenous defense system. Similarly, the present inventors found the oxidative stress-induced DNA damage was elevated in pig pericytes cultured with high-glucose medium. In this regard, the present inventors evaluated the antioxidant potency of PRDXs 5 and 6 and found that the addition of this protein to cell culture enhances cellular survival. It is difficult to assess the relative contribution of the PRDX system compared with other peroxidase; however, the present inventors can consider their distribution, turnover, and abundance. Catalase is an abundant, high-turnover enzyme, but it is localized to the peroxisomes and is relatively inefficient at low concentrations of hydrogen peroxide. PRDX is efficient for the removal of hydrogen peroxide at low concentrations because of its greater abundance and low Michaelis constant (<20 μM) (Chae et al., 1999, Diabetes Res Clin Pract. 45, 101-112). In various rat tissue, including lung, PRDX comprises about 1-10 μg/mg of soluble protein, and the cellular concentration of glutathione peroxidase is much lower than PRDX in most cells except hepatocytes (Chae et al., 1999, supra). Thus high abundance of PRDX in the cytoplasm allows it to be an important player in the detoxification of hydrogen peroxide. The present inventors have measured PRDX 5 and 6 levels in human retina samples. The levels of retinal PRDXs 5 and 6 were about 0.2-2.0 μg/mg and 1.0-6.0 μg/mg of soluble protein, respectively (unpublished data).

8-OHdG is a product of oxidative DNA damage and is a sensitive marker of increased oxidative stress (Morita et al., 2005, supra; Nishigori et al., 2005, supra; Sato et al., 2005, supra; Tarng et al., 2000, supra). Increased number of 8-OHdG positive cells cultured with high-glucose medium implicates that high glucose induces oxidative stress in pig pericytes. 8-OHdG levels are increased in the diabetic retina, which is inhibited by the same antioxidant therapy that inhibits diabetic retinopathy in rats (Kowluru and Odenbach, 2004, Diabetes. 53, 3233-3238). The present inventors have previously reported that PRDX 6 prevents high-glucose-induced cell death in human LECs overexpressing aldose reductase (AR) (Kubo et al., 2004, supra). The present inventors' findings revealed that PRDX 6 is a negative regulator of the death pathway induced in hyperglycemia (Kubo et al., 2004, supra). Because of these properties, PRDX could be an important molecule in the prophylaxis of hyperglycemia-induced complications. Thus, the therapies that inhibit ROS production via PRDXs 5 and 6 also may inhibit the pericyte loss in diabetic retinopathy. In cultured pig pericytes, the expressions of PRDX 5 mRNA and protein were down-regulated in pig pericytes cultured with medium containing 30 mM (30 G) D-glucose for 10 days. The depletion of PRDX 5 protein may induce the production of oxidative stress and cell death.

The present inventors' study shows that biologically active recombinant PRDX 5 and 6 proteins bearing the protein transduction domain TAT can be entered into cells and protects them from high glucose-induced cell apoptosis and ROS elevation. The applicability of this new approach has been demonstrated for inter/intramolecular targeting of TAT-fusion proteins capable of modulating mitochondrial function and cell survival (Kubo et al., 2008, supra; Shokolenko et al., 2005, DNA Repair (Amst). 4, 511-518). Also, the intravitreous injection of TAT-HA-PRDX 5 and 6 proteins can be transduced into rabbit retina (unpublished data of the department of ophthalmology in University of Fukui, Japan). Administration of intravitreous injection of TAT-linked PRDX 5 or 6 protein may be useful for the therapeutic approach in retinal eye disease.

In summary, administration of PRDXs 5 and 6 can inhibit apoptosis and oxidative damage to DNA in cultured pig pericytes. Thus, supplementation with PRDXs 5 and 6 may represent an achievable adjunct therapy to help a delay of the early progression of diabetic retinopathy.

Furthermore, as described above, oxidative damage to the retinal pigment epithelial cells (RPE) may play a role in some of the key features of age-related macular degeneration. As shown from the result of Example 6, delivery of PRDX 5 and PRDX 6 may protect RPE from oxidative stress in human RPE, and the delivery of PRDX 5 or 6 into RPE could be a promising approach in the prevention of early AMD.

### INDUSTRIAL APPLICABILITY

The present invention provides an agent for the prophylaxis or treatment of retinopathy or age-related macular degeneration and a method for the prophylaxis or treatment of retinopathy or AMD.

While this invention has been described with an emphasis upon preferred embodiments, it will be obvious to those of ordinary skill in the art that variations of the preferred embodiments may be used and that it is intended that the invention may be practiced otherwise than as specifically described herein. Accordingly, this invention includes all modifications encompassed within the spirit and scope of the invention as defined by the following claims.

All of the references cited herein, including patents, patent applications, and publications, are hereby incorporated in their entireties by reference.

This application is based on a U.S. provisional patent application No. 61/157,815 (filing date: Mar. 5, 2009), the contents of which are incorporated in .full herein by this reference.

1-20. (canceled)

21. An agent for the prophylaxis or treatment of retinopathy or macular degeneration comprising PRDX 5 or PRDX 6.

22. The agent of claim 21, wherein the retinopathy is diabetic retinopathy.

23. The agent of claim 21, wherein the PRDX 5 or PRDX 6 is fused with a protein transduction domain (PTD).

24. A method for preventing or treating retinopathy or macular degeneration, which method comprises administrating an effective amount of PRDX 5 or PRDX 6 to a subject in need thereof, thereby preventing or treating retinopathy or macular degeneration in the subject.

25. The method of claim 24, wherein the retinopathy is diabetic retinopathy.

26. The method of claim 24, wherein the PRDX 5 or PRDX 6 is fused with a protein transduction domain (PTD).

27. An agent for the prophylaxis or treatment of retinopathy or macular degeneration comprising a polynucleotide encoding PRDX 5 or PRDX 6.

28. The agent of claim 27, wherein the polynucleotide is incorporated into an expression vector.

29. The agent of claim 27, wherein the retinopathy is diabetic retinopathy.

30. The agent of claim 29, wherein the polynucleotide is incorporated into an expression vector.

31. The agent of claim 27, wherein the polynucleotide encodes a fusion protein of the PRDX 5 or PRDX 6 and a protein transduction domain (PTD).

32. The agent of claim 31, wherein the polynucleotide is incorporated into an expression vector.

33. A method for preventing or treating retinopathy or macular degeneration, which method comprises administrating an effective amount of a polynucleotide encoding PRDX 5 or PRDX 6 to a subject in need thereof, thereby preventing or treating retinopathy or macular degeneration in the subject.

34. The method of claim 33, wherein the polynucleotide is incorporated into an expression vector.

35. The method of claim 33, wherein the retinopathy is diabetic retinopathy.

36. The method of claim 35, wherein the polynucleotide is incorporated into an expression vector.

37. The method of claim 33, wherein the polynucleotide encodes a fusion protein of the PRDX 5 or PRDX 6 and a protein transduction domain (PTD).

38. The method of claim 37, wherein the polynucleotide is incorporated into an expression vector.
